(19) United States
(12) Patent Application Publication
(10) Pub. No.: US 2010/0113800 A1
(43) Pub. Date: May 6, 2010

(54) Formation of Carbon-nitrogen bond

(75) Inventors: Christelle Mauger, Oullins (FR); Gerard Mignani, Lyon (FR); Roland Jacquot, Francheville (FR)

Correspondence Address: BUCHANAN, INGERSOLL & ROONEY PC, POST OFFICE BOX 1404, ALEXANDRIA, VA, 22313-1404, US

(73) Assignee: SHASUN PHARMA SOLUTIONS LIMITED, NORTHUMBERLAND (GB)

(21) Appl. No.: 11/990,012

(22) PCT Filed: Aug. 4, 2006
(86) PCT No.: PCT/IB2006/002146

§ 371 Date: Jan. 13, 2010

(30) Foreign Application Priority Data
Aug. 5, 2005 (FR) 05 08377

(51) Int. Cl. C07D 233/00

(52) U.S. Cl. 548/335.1

(57) ABSTRACT

The present invention relates to a process to create a carbon-nitrogen bond by the reaction of an aromatic compound carrying at least one electro attractive group with one nitrogenous heterocyclic type nucleophile compound. The invention aims, in particular, at a link reaction between an aromatic compound carrying at least one electro attractive group and an imidazole heterocycle type. The invention process, consisting of the reaction of an aromatic compound carrying one leaving group and at least one electro attractive group and a nitrogenous heterocyclic type nucleophile compound comprising a N—H pattern likely to substitute the leaving group, thereby creating a carbon-nitrogen bond in the presence of a copper catalyst, of a base in an organic solvent, is characterized by the fact that the said nitrogenous heterocycle reacts with an aromatic type electrophile compound carrying one leaving group (Y) chosen from bromine, chlorine or sulfonic ester and at least one electro attractive group (Z1), in the presence of a copper catalyst, of a mineral or organic base, of an extracting agent selected from aliphatic diamines, aliphatic amino alcohols and diols in a polar aprotic organic solvent with a dielectric constant less than about 20 and a basicity such that its “donor number” is less than about 25.

The aim of this invention is a process creating a carbon-nitrogen bond by the reaction of an aromatic compound carrying at least one electro attractive group and one nitrogenous heterocyclic type nucleophile compound.

The invention aims, in particular, at a link reaction between an aromatic compound carrying at least one electro attractive group and an imidazole heterocycle type.

The structures comprising an aromatic ring bonding like benzene and a heterocyclic nitrogenous ring like for example an imidazole, come together in several molecules used in the pharmaceutical domain:

The N-arylation of a nitrogenous heterocycle is described in the documentation.

J. C. Antilla et at (J. Org. Chem 69, 5578-5587 (2004)) have thus described the reaction of a halogenobenzene and of a heterocyle, like pyrrole, pyrazole, indazole, imidazole and triazole, reaction in the presence of an iodide containing copper, base (potassium phosphate, potassium or cesium carbonate) and in an organic solvent (toluene, dioxane, DMF).

However, it is proven that the said link reaction is difficult when the electrophile, that is, halogenobenzene, relies on an aromatic nucleus, an electro attractive group that may affect one of the kinetic steps of the catalytic cycle, which in turn will make the link reaction more difficult.

To overcome this problem, the aforesaid publication proposes that an iodobenzene, which is far more reactive than a bromobenzene, is used.

Thus, the link reactions between an iodobenzene carrying an electroattractive group and a nitrogenous heterocycle is described.

Yet, from an industrial point of view, it is not possible to consider an iodine type halogenobenzene because this product is not only reactive but also thermically unstable and sensitive to U.V. radiation.

One aim of the invention is to provide a process that enables the achievement of the bonding in better conditions.

A subject matter of this invention, is the discovery of a process for forming a carbon-nitrogen bond by reacting an aromatic compound carrying a leaving group and at least one electro attractive group and one nitrogenous heterocyclic type nucleophile compound comprising a N—H pattern likely to substitute the leaving group, thereby creating a carbon-nitrogen bond, in the presence of a copper catalyst and of a base in an organic solvent; characterized by the fact that the said nitrogenous heterocycle reacts with an aromatic type electrophile compound carrying one leaving group (Y) chosen from bromine, chlorine or sulfonic ester and at least one electro attractive group (Z₁), in the presence of a copper catalyst, a mineral or organic base, an extracting agent selected from aliphatic diamines, aliphatic amino alcohols and diols in a polar aprotic organic solvent with a dielectric constant less than about 20 and a basicity such that its “donor number”(“nombre donneur”, in the French language) is less than about 25.

The applicant has founded that it is possible to carry out a bonding when an aromatic electrophile compound consisting of one or more electro attractive groups, and a nitrogenous heterocycle come into contact, as long as the reaction is carried out in the presence of a copper catalyst and an alkaline cation extracting agent as defined earlier and, and in an aprotic organic solvent, with certain polarity and basicity characteristics.

### Nucleophile Compound.

<<Nitrogenous heterocyclic type nucleophile compound>> means a monocyclic or polycyclic heterocycle in which at least one of the carbon atoms of the ring is replaced by a N—H pattern.

To be more precise, it satisfies the general formula:

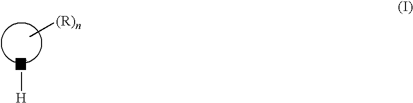

in the said formula(I):
the ring symbolizes the remainder of a ring forming the whole or part of the heterocyclic, saturated, unsaturated or aromatic, monocyclic or polycyclic system,
R, identical or different, represents a hydrogen atom, or a substituent,
n represents the number of substituents in the cycle.

The invention is applied especially to the monocyclic heterocyclic compounds that satisfy formula (I) in which the ring represents a heterocycle, saturated or otherwise, or aromatic containing particularly 5 or 6 atoms in the ring that can, in turn, contain 1, 2 or 3 nitrogen atoms of which at least one is a nucleophile pattern, like NH.

The cycle can also represent a defined heterocyclic polycyclic compound as being, constituted by at least 2 aromatic or non aromatic heterocycles containing at least one nitrogen atom in each cycle and forming ortho-, or ortho- and perk, condensed systems between them, or a group constituted by at least one aromatic or non-aromatic carbocycle and at least one aromatic or non-aromatic nitrogenous heterocycle forming ortho-, or ortho- and peri, condensed systems between them.

It is further possible to start from a substrate resulting from the linking of a saturated, unsaturated or aromatic heterocycle as mentioned earlier, and a saturated, unsaturated or aromatic carbocycle. Carbocycle generally means a cycloaliphatic or aromatic ring having between 3 and 8 carbon atoms, preferably 6.

It is to be noted that the carbon atoms of the heterocycle can possibly be substituted, either completely or partially, by the R groups only.

The number of substituents present in the ring depends on the number of atoms in the ring and the presence or absence of the unsaturations in the ring.

The maximum number of substituents that are likely to be carried by a ring is easily determined by those skilled in the art.

Generally, the number of substituents is at the most equal to the number of carbon atoms present in the heterocycle.

In the formula (I), n is a number, preferably equal to 0 or 1.

Some examples of substituents are given below but this list is not intended to be limitative.

The identical or different R group or groups, represent, preferably, one of the following groups:

an alkyl, cycloalkyl, phenyl group,

an alkoxy group,

a group or function like ester, nitrile, nitro, trifluoromethyl.

In this text, <<alkyl>> means a linear or branched hydro carbonated chain in C₁₋₁₂ , preferably in C₁₋₄. Examples of preferred alkyl groups are especially methyl, ethyl, propyl, isopropyl, butyl, isobutyl, t-butyl.

<<Cycloalkyl>> is a cyclic, monocyclic hydro carbonated group in C₅₋₈, preferably, a cyclopentyl or cyclohexyl group.

<<Alkoxy>> is an alkyl-oxy group that comprises 1 to 6 atoms in the alkyl chain.

<<Ester>> is an alkyl or cycloalkyl ester as defined earlier.

This invention is applicable, in particular; to compounds satisfying the formula (I) wherein the R group or groups represent in particular an alkyl or alkoxy group, preferably, methyl group.

Especially in formula (I), the ring optionally substituted represents one of the following rings:
a monocyclic heterocycle comprising one or several heteroatoms:

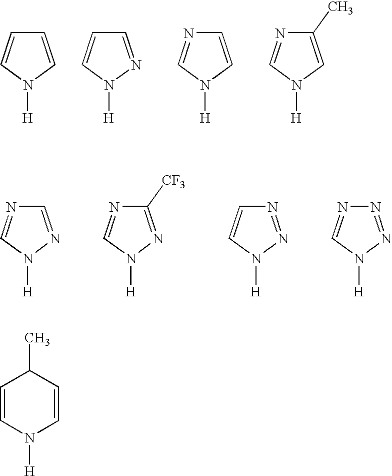

a bi-cycle comprising a carbocycle and a heterocycle comprising one or several heteroatoms:

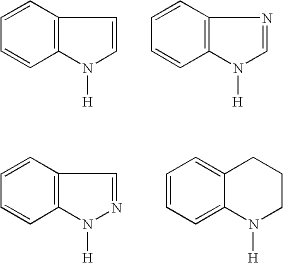

a tri-cycle comprising at least one carbocycle or a heterocycle comprising one or several heteroatoms:

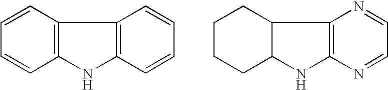

For heterocyclic compound examples, only those that satisfy the formula (I) wherein the ring represents a ring such as pyrazole, pyrazolidine, imidazole, imidazolidine, indole, pyrrolidine, pyrrole, triazole, are preferentially used.

Among the compounds satisfying formula (I), those that are of imidazole type and that satisfy the following formula are more preferentially used:

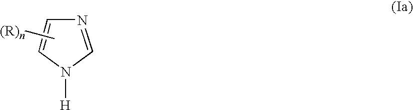

in the said formula, R and n have the same signification as mentioned earlier.

n is preferably equal to 1.

R, preferably represents a C₁-C₄ alkyl group, preferably a methyl group,

The nitrogenous heterocycle type nucleophile is advantageously 4-methylimidazole.

### Aromatic Electrophile Compound.

The electrophile compound concerns an aromatic compound satisfying this formula:

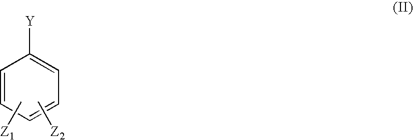

in the said formula,
Z₁, Z₂, identical or different, represent a hydrogen atom or a substituent,
at least one of the Z₁, Z₂ group is an electro attractive group,
Y represents a leaving group chosen from bromine, chlorine or a sulphonic ester group following the formula —OSO₂—R₁, wherein R₁ is a hydro carbonated group.

In the sulphonic ester group formula, R₁ is a hydro carbonated group of any type. Nevertheless, given that Y is a leaving group, it is interesting from an economical viewpoint that R₁ should be of simple nature and represent, in particular, a linear or branched alkyl group having between 1 and 4 carbons atoms, preferably, a methyl or ethyl group but it can also represent a phenyl, tolyl or trifluoromethyl group. Among the Y groups, the preferred group is a triflate group which corresponds to an R₁ group representing a trifluoromethyl group.

As preferred leaving groups, it is preferable to choose a bromine atom.

In formula (II), Z₁ represents at least one electro attractive group.

By <<electro attractive group>>, we mean the groups defined by H. C. BROWN in a book by Jerry MARCH—Advanced Organic Chemistry, 4^{th} edition, John Wiley and Sons, 1992, chapter 9, pp. 273-292.

Ester, nitrile, nitro, trifluoromethyl and preferably, nitro, trifluoromethyl groups may be given as examples of electro attractive groups.

The other Z₂ group can be a hydrogen atom, another electro attractive group or even an electro-donor.

Refer to the above mentioned book for the definition of <<electro-donor group>>.

Some specific examples of electro-donor groups are alkyl, cycloalkyl, alkoxy, amino or amido groups substituted by identical or different alkyl or cycloalkyl groups.

In formula (II), the Z₁ and Z₂ groups are normally in the meta position with respect to the leaving group Y.

An aromatic compound satisfying formula (II) consisting of at least one electro attractive group is provided preferentially in the process of the invention.

An aromatic compound satisfying formula (II) consisting of two electro attractive groups is also provided in the process of the invention.

Among the aromatic electrophile compounds satisfying formula (II), only those that satisfy the following formula are more specifically used:

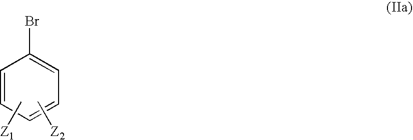

in the above formula, Z₁ and Z₂ have the same meaning as given earlier and at least one of the two Z₁ and Z₂ groups is an electro attractive group.

In formula (IIa), Z₁ and Z₂ may both be electro attractive groups.

Below given are examples of aromatic electrophile compounds that have been used: 4-bromotoluene, 4-bromotrifluoromethylbenzene, 4-bromonitrobenzene, 4-bromoanisole, 4-bromoaniline, 4-bromofluorobenzene, 3-bromotrifluoromethylbenzene, 3-bromoanisole, 3-bromonitrobenzene, 3-bromoaniline, 2-bromoanisole, 2-bromonitrobenzene, 2-bromotrifluoromethylbenzene, 1-bromo-3,5-difluorobenzene, 3,5-bis(trifluoromethyl)bromo-benzene, 1-bromo-3,5-dimethoxybenzene, Ie 1-bromo-3,5-di-tert-butylbenzene, Ie 3-amino-5-bromotrifluoromethylbenzene, Ie 1-bromo-3,5-dimethylbenzene.

In the invention process, nitrogenous nucleophile compound preferably satisfying formula (I) and more preferably (Ia), is made to react with an aromatic electrophile compound that satisfies formula (II), preferably formula (IIa), in the presence of an efficient amount of a copper catalyst, a base, an extracting agent and a solvent medium.

The amount of aromatic electrophile compound used, described in respect to the nucleophile compound amount, is generally close to stoechimetry. Therefore, the ratio between the number of moles of the aromatic electrophile compound and the number of moles of the nucleophile compound, often varies between 0.9 and 1.5, preferably between 1 and 1.2.

### Catalyst.

The catalysts used in the invention process are known products.

Some examples of catalysts that are likely to be used are copper metal (0) or organic, or inorganic, compounds of copper (I) or of copper (II).

As examples of catalysts used for the invention, may be cited as copper compounds, cuprous bromide, cupric bromide, cuprous iodide, cuprous chloride, cupric chloride, basic cupric carbonate (II), cuprous nitrate, cupric nitrate, cuprous sulphate, cupric sulphate, cuprous sulphate, cuprous sulfite, cuprous oxide, cuprous acetate, cupric acetate, cupric trifluoromethylsulfonate, cupric hydroxide, copper methylate (I), copper methylate (II), chlorocupric methylate satisfying the formula ClCuOCH₃d.

Generally, a copper (I) based catalyst is chosen, preferably cuprous iodide, bromide or chloride or more preferably cuprous iodide.

The quantity of the catalyst used that is expressed in molar ratio between the number of moles of the copper catalyst given in copper and the number of moles of the aromatic electrophile compound, generally varies between 0.01 and 0.2, preferably between 0.01 and 0.1.

### Base.

A base which has the function to stop the hydracid formed during the reaction, is also provided in the invention process.

The characteristic of the base is that it has a pKa that is at least more than or equal to 4, or equal to 8, preferably between 4 and 14, between 8 and 11, rather between 4 and 10.

The pKa is defined as the ionic dissociation constant of the acid/base couple when water is used as the solvent.

In order to choose a base with a pKa as defined by the invention, Handbook of Chemistry and Physics, 66^{th} edition, p. D-161 and D-162 may be consulted, among others.

Among the bases that can be used, mineral bases like carbonates, hydrogencarbonates, phosphates and polyphosphates, borates, alkaline metal silicates, preferably sodium or potassium; alkaline earth silicates, preferably calcium, barium or magnesium ; transition metals, preferably zinc and copper are cited among others.

The common examples cited are sodium and potassium carbonate, potassium and sodium hydrogen carbonate, potassium phosphate, zinc or copper carbonate.

Organic bases are also suitable, like tertiary amines and picolines, in particular, triethylamine, tri-n-propylamine, tri-n-butylamine, methyldibutylamine, methyldicyclohexylamine, ethyldiisopropylamine, N,N-diethylcyclohexylamine, pyridine, dimethylamino-4-pyridine, N-methylpiperidine, N-ethylpiperidine, N-n-butylpiperidine, 1,2-dimethylpiperidine, 2-picoline, 4-picoline.

Among the bases, potassium carbonate is preferred.

The base is advantageously used in solid form so that not too much water is added to the reacting medium.

Water must not be more than 10% in volume of the reacting mixture but can advantageously be between 2 and 3%.

The quantity of base used must be at least equal to the quantity corresponding to number of NH moles of the nitrogenous nuclepophile compound. It is also possible to use a corresponding excess, that is, 4 times the stoechiometric quantity. To advantage, the quantity of the base varies from the stoechiometric quantity up to 4 times the stoechiometric quantity. If the base used presents two basic sites, for example, as potassium carbonate, the ratio between the number of moles of the base and the number of moles of the nitrogenous nucleophile compound varies advantageously, between 0.5 and 2 and is preferably around 0.5.

### Solvent.

Several requirements are to be fulfilled when choosing an organic solvent.

A first characteristic of the organic solvent is that it must be aprotic and stable in a reacting medium.

Aprotic solvent means a solvent that does not have protons to release, according to the Lewis theory.

Solvents that are not stable in the reacting medium and which disintegrate either by oxidation or hydrolysis are not included in this invention. For example, ester, aldehyde primary and secondary amines and alcohol type solvents are examples of reacting solvents that cannot be used in this invention.

Organic solvents suitable for use in this invention process should satisfy certain demands of their polarity and basicity level characterised by the donor number.

One group of organic solvents that are well suitable for use in the invention process is organic solvents that are less polar and alkaline.

According to the invention, an organic solvent that presents a dielectric constant less than about 20 is chosen. The lower limit does not present any critical character. It is better to use an organic solvent having a weak dielectric constant, preferably between 2 and 15.

The organic solvent having the above mentioned polarity characteristics should also satisfy certain basicity conditions. Actually, the said solvent should not be too alkaline. In order to determine if a solvent satisfies this need, its basicity can be estimated according to its <<donor number>>.

An organic solvent is chosen with a donor number that lies between 4 and 25. An organic solvent with a donor number between 7 and 20 is preferably chosen.

In order to determine if the organic solvent satisfies the conditions of the dielectric constant that are explained above, the book, Techniques of Chemistry, II—Organic solvents—from p. 536, 3^{rd} edition (1970) may be referred, among others.

For the basicity requirements of an organic solvent to be used, it has to be remembered that the “donor number” assigned in a short form, DN, gives an indication of the solvent's nucleophile character and reveals its capacity to give its doublet.

In Christian REINHARDT's book, [Solvents and Solvent Effects in Organic Chemistry—VCH p. 19 (1988)], “donor number” is defined as the negative of (−ΔH) the enthalpy (Kcal/mol) of the interaction between the solvent and antimony pentachloride, in a diluted dichloroethane solution.

Aliphatic, cycloaliphatic or aromatic ether-oxides and in particular diethyl oxide, dipropyl oxide, diisopropyl oxide, dibutyl oxide, methyl and tertiobutyl oxide, dipentyl oxide, diisopentyl oxide, ethylneglycol dimethylether (or 1,2-dimethoxyethane), diethylneglycol dimethylether (ou 1,5-dimethoxy-3-oxapentane), anisole, 1,2-dimethoxybenzene, 1,3-dimethoxybenzene, 1,4-dimethoxybenzene, biphenyl oxide and or benzyl oxide, 1,2-diphenoxyethane, dioxane, tetrahydrofurane may be given as examples of aprotic polar organic solvents that satisfy the aforesaid basicity requisites, likely to be used in the invention process.

Anisole is the preferred solvent.

A mixture of solvents may also be used.

The quantity of the organic solvent used is determined in order that the electrophile molar concentration in the organic solvent is preferably, between 1 and 5 M, and more preferably, between 1 and 2M.

### Extracting Agent

The extracting agent that intervenes in the invention process must be able to form a complex with the base cation.

In accordance to the invention process, the following different extractors are considered: aliphatic diamines, aliphatic aminoalcohols and aliphatic diols.

<<Aliphatic diamine>>, in this text, means a group of aminos, optionally substituted, which are linked to an aliphatic chain consisting of 1 to 6 carbon atoms.

The preferred extracting agents consist of an aliphatic chain carrying to two amino groups.

They may be represented by the following formula:

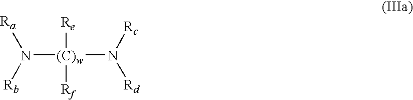

in the said formula:
Rₐ, R_{b}, R_{c}, R_{d}, identical or different, represent a hydrogen atom or an alkyl group,
Rₑ, R_{f}, identical or different, represent a hydrogen atom, an alkyl group or a phenyl group,
w lies between 0 and 6.

The preferred extracting agents are those of formula (IIIa) wherein Rₐ, R_{b}, R_{c}, R_{d}, identical or different represent a hydrogen atom or an alkyl group having 1 to 4 carbon atoms and more preferably, Rₐ, R_{b}, R_{c}, R_{d}, represent a hydrogen atom.

The preferred extracting agents are those of formula (IIIa) wherein Rₑ and R_{f}, identical or different represent a hydrogen atom or an alkyl group having 1 to 4 carbon atoms and more preferably, Rₑ and R_{f}, represent a hydrogen atom.

As far as w is concerned, preferably w lies between 2 and 4 and is, preferably equal to 2.

1,2-diaminoethane, 1,3-diaminopropane, 2,3-diaminobutane, N,N,N′,N′-tetramethyl-1,2-diaminoethane, N,N′-diisopropyl-1,2-diaminoethane, N,N′-dimethyl-1,2-diaminoethane, 2,3-diamino-2,3-dimethylbutane, 2,3-diamino-2,3-diphenylbutane may be cited as examples of extracting agents suitable for use in the invention process.

In accordance with the invention process, as extracting agents, a diol or an aliphatic aminoalcohol preferably satisfying the following formula may also be considered:

HO-Ψ-G (IIIb)

in the said formula:
Ψ represents a linear or branched aliphatic chain having 2 to 20 carbon atoms,
G represents one of the following functional groups:
   OH
   NH₂
   NHRₐ
   NRₐR_{b}

in the said groups, Rₐ, R_{b}, identical or different, represent an alkyl group.

To be more precise, in the formula (IIIb), Ψ represents a linear or branched aliphatic divalent group, preferably having 2 to 20 carbon atoms.

The hydro carbonated chain may possibly be interrupted by a heteroatom or a functional group, preferably, oxygen, NH or NHRₐ.

The hydro carbonated chain may optionally carry one or several substituents, for example another OH group. There may also be other substituents in such a way that they don't react in the reaction conditions.

Therefore, the Ψ group represents a ethylene, propylene, isopropylene, isobutylene, pentylene, propanetri -1,2,3-yle, 3-oxypentylene, 3-iminopentylene group.

As for functional group G, it may be a hydroxyl group, an amino group or a substituted amino group wherein the groups Rₐ, R_{b}, identical or different, represent an alkyl group having 1 to 4 carbon atoms and even more preferably, Rₐ, R_{b}, represent a methyl or ethyl group.

In the formula (IIIb), G represents preferably a hydroxyl group or an amino group.

The following may be cited as examples for extracting agents that are suitable for use in the invention process:
diol type extracting agents like:
   ethyleneglycol,
   1,3-propanediol,
   1,2-propanediol,
   1,2-butanediol,
   1,4-butanediol,
   1,3 butanediol,
   diethyleneglycol,
   glycerol,
aminoalcohol type extracting agents like:
   ethanolamine,
   N,N-dimethylethanolamine,
   1-amino-2-propanol,
   3-amino-1-propanol,
   2-amino-1-propanol,
   2-amino-1-butanol,
   4-amino-1-butanol,
   4-amino-2-butanol,
   2-(ethylamino)ethanol,
   2-amino-1,3-propanediol,
   3-amino-1,2-propanediol,
   2-amino-2-methyl-1,3-propanediol,
   diethanolamine,
   N-methyldiethanolamine,
   3-methylamino-1,2-propanediol,
   2-amino-1,3-butanediol.

The quantity of extracting agent, expressed by the ratio between the number of moles of the extracting agent and the number of moles of the base, varies between 0.1 and 1.0, preferably between 0.1 and 0.5.

As for the reaction temperature of the reaction between the nucleophile compound and the electrophile compound, it is advantageously chosen in such a way that the reagents are in a liquid state.

The linking reaction takes place at a temperature between 80° C. and 170° C., preferably, between 120° C. and 160° C., and even better around 140° C.

The linking reaction is generally implemented under atmospheric pressure but higher pressures such as those that can go up to 10 Bar may also be used.

In accordance with a preferred alternative of the invention process, the invention process is carried out under a controlled atmosphere of inert gases. An atmosphere of rare gases may be established, preferably argon, but it is more economical to use nitrogen.

Looking at it practically, the reaction is very simple to implement.

A first embodiment of the reagents consists of introducing all the reagents, including the solvent, in any order.

Another embodiment and which is preferred is to first pre-form the catalyst and then add it to the already introduced reagents.

An embodiment consists of pre-forming the catalyst which means that the metallic element Cu may either be brought already in a complex form comprising one of the extracting agents mentioned earlier, or the metallic element Cu and the extracting agent are separately introduced in the medium.

The metallic complex may also be prepared at the beginning of the reaction from a nitrogenous heterocycle which acts as a ligand and a compound carrying the metallic element Cu.

The catalytic complex may be obtained, for example, by heating the compound carrying the Cu element and the extracting agent at 20° C. to 100° C., preferably at 20° C., in the solvent or solvents used for the reaction.

After bringing the catalyst in contact with the reagents, the reaction medium is braught to the chosen temperature of the reaction.

The progress of the reaction is monitored by following the disappearance of the compound carrying the leaving group.

At the end of the reaction, a binding product is obtained which may be symbolised by the following formula:

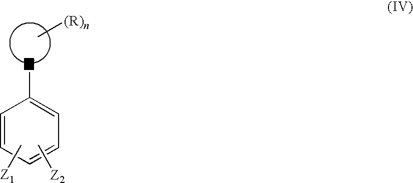

in the said formula, R, Z₁, Z₂ and n have the earlier given signification.

The copper catalyst is eliminated by washing it in a watery ammonia solution (about 20% by weight).

The liquid and organic phases are separated.

The obtained product will be in an organic phase.

The organic phase is acidified with the help of a hydrochloric or sulphuric acid solution in such a quantity that the ratio between the number of nitrogen atoms (1 or 2) of the obtained product and the number of protons brought by the acid is equal to about 1.

The aqueous and organic phases are separated.

The desired product is in aqueous phase.

A base is added, preferably soda so that the ratio between the number of nitrogen atoms and number of OH⁻ brought from the base is equal to about 1.

The obtained product is precipitated in aqueous phase and separated using the conventional techniques of solid/liquid separation, preferably by filtration. The obtained product may be purified by recrystallization, for example, in aliphatic (hexane, heptane), cycloaliphatic (cyclohexane, methylcyclohexane) or ether aliphatic (diisopropylether) hydrocardbon.

The invention process enables achieving a bonding with an electrophile carrying two electro-attractive groups, with very good yield.

Examples of embodiments of the invention are given below. These examples are only indicative and are not intended to be limitative.

In the examples, the rate of transformation corresponds to the ratio between the number of moles of the transformed electrophile compound and the number of moles of the introduced electophile compound.

The yield given in the examples corresponds to the ratio between the number of moles of the formed product and the number of moles of the introduced electrophile compound.

Before listing in detail the different examples, first the protocol that has always to be followed is given:

### Protocol:

In a glass reactor of 100 l, equipped with magnetic stirring, all the reagents are introduced under an argon flow : aromatic bromine derivate (1 eq), 4-methylimidazole (1.22 eq), ethylene glycol (0.05 eq), potassium carbonate (0.6 eq) and copper iodide l) (0.02 eq).

The reactor is then several times flushed under argon, then under reduced pressure (about 30 mm of mercury).

Anisole solvent (1.76 eq) is then added.

The mixture is then heated to about 140-145° C., for 22 hours, under argon flow, vigourously shaken all the time.

The reactive medium is set to cool to room temperature and then filtered.

Water is added and the medium is carefully acidified using concentrated hydrochloric acid up to pH=1.

The different phases are separated and the aqueous phase is preserved.

Then, ammonia in aqueous solution (28%) is added up to pH=10 while shaking it, which leads to the precipitation of the products in a white solid form surrounded by an oily phase.

The aqueous phase is extracted with toluene and the organic phase recovered is dried on magnesium sulphate.

The crude products are obtained by evaporation process in vaccum at 65° C. under 40 mbars and are presented in the form of oils or solids of a pale yellow to deep brown colour.

### EXAMPLES 1 TO 13

In this series of samples, the 4-methylimidazole is made to react with different monosubstituted electrophile compounds, that is, bromoaromatic compounds like those specified in table (I).

The results obtained are given in the following table:

TABLE (I)

| | | | Transformation | Overall |
|---|---|---|---|---|
| | | | rate of the | isolated |
| Ref. | | | electrophile | crude |
| Ex. | Electrophile Compound | Product obtained | compound (%) | yield (%) |
| 1 | 4-bromotoluene | N-(4-methyl-phenyl)- | 92 | 65 |
| | | 4-methylimidazole | | |
| 2 | 4-bromotrifluoromethyl- | N-(4-trifluoromethyl- | 100 | 65 |
| | benzene | phenyl)-4- | | |
| | | methylimidazole | | |
| 3 | 4-bromonitrobenzene | N-(4-nitro-phenyl)-4- | 100 | 65 |
| | | methylimidazole | | |
| 4 | 4-bromoanisole | N-(4-methoxy- | 84 | 62 |
| | | phenyl)-4- | | |
| | | methylimidazole | | |
| 5 | 4-bromoaniline | N-(4-amino-phenyl)- | 93 | 55 |
| | | 4-methylimidazole | | |
| 6 | 4-bromofluorobenzene | N-(4-fluoro-phenyl)- | 99 | 70 |
| | | 4-methylimidazole | | |
| 7 | 3-bromotrifluoromethyl- | N-(3-trifluromethyl- | 94 | 65 |
| | benzene | phenyl)-4- | | |
| | | methylimidazole | | |
| 8 | 3-bromoanisole | N-(3-methoxy- | 95 | 65 |
| | | phenyl)-4- | | |
| | | methylimidazole | | |
| 9 | 3-bromonitrobenzene | N-(3-nitro-phenyl)-4- | 100 | 55 |
| | | methylimidazole | | |
| 10 | 3-bromoaniline | N-(3-amino-phenyl)- | 55 | 50 |
| | | 4-methylimidazole | | |
| 11 | 2-bromoanisole | N-(2-methoxy- | 76 | 60 |
| | | phenyl)-4- | | |
| | | methylimidazole | | |
| 12 | 2-bromonitrobenzene | N-(2-nitro-phenyl)-4- | 82 | 50 |
| | | methylimidazole | | |
| 13 | 2-bromotrifluoromethyl- | N-(2-trifluoromethyl- | 55 | 20 |
| | benzene | phenyl)-4- | | |
| | | methylimidazole | | |

### EXAMPLES 14 TO 19

In these series of trials, 4-methylimidazole is made to react with different disubstituted electrophile compounds, namely bromoaromatic compounds like those specified in table (II).

The results obtained are given in the following table:

TABLE (II)

| | | | Transformation | Overall |
|---|---|---|---|---|
| | | | rate of the | isolated |
| Ref. | | | electrophile | crude |
| ex. | Electrophile compound | Product obtained | compound (%) | yield (%) |
| 14 | 1-bromo-3,5-difluorobenzene | N-(3,5-difluoro- | 100 | 70 |
| | | phenyl)-4- | | |
| | | methylimidazole | | |
| 15 | 3,5-bis(trifluoromethyl)bromo- | N-(3,5- | 100 | 60 |
| | benzene | bis(trifluoromethyl) | | |
| | | phenyl)-4- | | |
| | | methylimidazole | | |
| 16 | 1-bromo-3,5-dimethoxybenzene | N-(3,5-dimethoxy- | 85 | 70 |
| | | phenyl)-4- | | |
| | | methylimidazole | | |
| 17 | 1-bromo-3,5-di-tert- | N-(3,5-di-tert-butyl- | 54 | 20 |
| | butylbenzene | phenyl)-4- | | |
| | | methylimidazole | | |
| 18 | 3-amino-5- | N-(3-amino-5- | 78 | 60 |
| | bromotrifluoromethylbenzene | trifluoromethyl- | | |
| | | phenyl)-4- | | |
| | | methylimidazole | | |
| 19 | 1-bromo-3,5-dimethylbenzene | N-(3,5-dimethyl- | 96 | 70 |
| | | phenyl)-4- | | |
| | | methylimidazole | | |

1- A process for forming a carbon-nitrogen bond by reaction of an aromatic compound carrying a leaving group and at least one electro attractive group and a nitrogenous heterocyclic type nucleophile compound comprising an N—H pattern able to substitute the leaving group, whereby a carbon-nitrogen bond is created, in the presence of a copper catalyst, of a base, in an organic solvent, which is characterized in that the said nitrogenous heterocycle reacts with an aromatic type electrophile compound carrying a leaving group (Y), chosen from bromine, chlorine or sulfonic ester, and at least one electro attractive group (Z₁), in the presence of a copper catalyst, a mineral, or organic, base, an extracting agent chosen from the aliphatic diamines, aliphatic aminoalcohols and diols, and in an aprotic polar organic solvent having a dielectric constant less than about 20 and having such a basicity that it possessed a “donor number” less than about 25.

2- The process of claim 1 wherein the nitrogenous heterocyclic type electrophile compound satisfies the general formula: 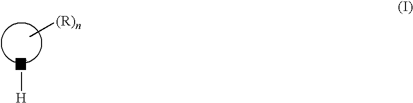
in the said formula:
the ring represents the whole or part of a saturated, unsaturated or aromatic, monocyclic or polycyclic heterocyclic system,
R, identical or different, represents a hydrogen atom or a subtituent,
n represents the number of Substituents in the cycle.

3- The process according of claim 1 or 2, wherein nitrogenous type nucleophile heterocyclic compound satisfies formula (I) wherein the ring represents:
a heterocycle which is saturated or not, or aromatic, preferably comprising 5 to 6 atoms in the ring, that may comprise 1, 2 or 3,nitrogen atoms, among which at least one is a nucleophile pattern like NH,
a polycyclic heterocyclic compound constituted by at least 2 heterocycles, aromatic or not, containing at least one nitrogen atom in each ring and forming ortho-, or ortho- and peri-, condensed systems between them, or by a group constituted by at least one carbocycle, aromatic or not, and at least one nitrogenous heterocycle, aromatic or not, forming ortho- or ortho- and peri-condensed systems between them,
a chain of a saturated, unsaturated or aromatic heterocycle as mentioned above and of a saturated, unsaturated or aromatic carbocycle.

4- The process of any of claims 1 to 3, wherein the nitrogenous heterocyclic type nucleophile compound matches formula (I) wherein the ring represents a heterocycle chosen among the following heteroycycles: pyrazole, pyrazolidine, imidazble, imidazolidine, indole, pyrrolidine, pyrrole, triazole.

5- The process of any of claims 1 to 4, wherein the nitrogenous heterocycle type nucleophile compound matches formula (I) wherein the R group or groups, identical or different, represent:
an alkyl, cycloalkyl, phenyl group,
an alkoxy group,
a group or function such as ester, nitrile, nitro, trifluoromethyl.

6- The process of any of claims 1 to 5, wherein the nitrogenous heterocyclic type nucleophile compound matches the formula (I) wherein n is a number equal to 0 or 1.

7- The process of any of claims 1 to 6, wherein the nitrogenous heterocyclic type nucleophile compound satisfies the following formula: 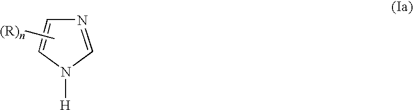
in which formula, R and n have the same signification as given in one of the claims 2, 5 and 6.

8- The process of claim 7, wherein n is equal to 1 and R represents an alkyl group C₁-C₄, preferably, a methyl group.

9- The process of anyone of claims 1 to 8, wherein the aromatic electrophile compound has the following formula (II): 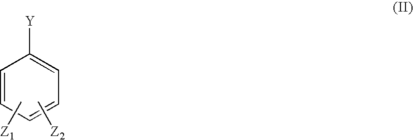
in the said formula,
Z₁, Z₂, identical or different, represent a hydrogen atom or a substituent,
at least one of the groups Z₁, Z₂ is an electro attractive group,
Y represents a leaving group chosen from bromine, chlorine or a sulfonic ester group with the formula —OSO₂—R₁, wherein R₁ is a hydrocarbon group.

10- The process of claim 9, wherein the aromatic electrophile compound satisfies formula (II) wherein Y is a sulfonic ester group with the formula —OSO₂—R₁ in which R₁ is a linear or branched alkyl group having 1 to 4 carbon atoms, preferably, a methyl or ethyl group, phenyl or tolyl group or a trifluoromethyl group.

11- The process of either of claims 9 and 10, wherein the aromatic electrophile compound satisfies formula (II) wherein Z₁ represents an ester, nitrile, nitro, trifluoromethyl group.

12- The process of any of claims 9 to 11, wherein the aromatic electrophile compound satisfies formula (II) wherein Z₂ is a hydrogen atom, another electro attractive group or even a electro-donor group.

13- The process of any of claims 9 to 11, wherein the aromatic electrophile compound satisfying formula (II) wherein Z₂ is an electro attractive group having the signification of Z₁ as given in claim 11.

14- The process of any of claims 9 to 11, wherein the aromatic electrophile compound satisfies formula (II) wherein Z₂ is an electro-donor group such as an alkyl, cycloalkyl, alkoxy, amino or amido group substituted by identical or different alkyl or cycloalkyl groups.

15- The process of any of claims 9 to 14, wherein the aromatic electrophile compound satisfies the following formula: 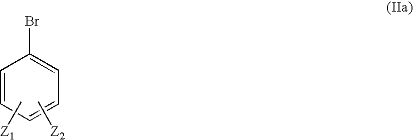
in the said formula, Z₁ and Z₂ have the same signification as given earlier in claims 11 to 14 and at least one of the two groups Z₁ and Z₂ is an electro attractive group.

16- The process of any of claims 9 to 15, wherein the aromatic electrophile compound satisfies formula (II) or (IIa) in which the groups Z₁ and Z₂ are in a meta position as compared to the leaving group.

17- The process of any of claims 9 to 16, wherein the aromatic electrophile compound satisfies formula (II) or (IIa) in which the groups Z₁ and Z₂ are both electro attractive groups.

18- The process of any of claims 9 to 17, wherein the aromatic electrophile compound is chosen from 4-bromotoluene, 4-bromotrifluoromethylbenzene, 4-bromonitrobenzene, 4-bromoanisole, 4-bromoaniline, 4-bromofluorobenzene, 3-bromotrifluoromethylbenzene, 3-bromoanisole, 3-bromonitrobenzene, Ia 3-bromoaniline, 2-bromoanisole, 2-bromonitrobenzene, 2-bromotrifluoromethylbenzene, 1-bromo-3,5-difluorobenzene, 3,5-bis(trifluoromethyl)bromo-benzene, 1-bromo-3,5-dimethoxybenzene, 1-bromo-3,5-di-tert-butylbenzene, 3-amino-5-bromotrifluoromethylbenzene, 1-bromo-3,5-dimethylbenzene.

19- The process of any of claims 1 to 18, wherein nitrogenous heterocyclic type nucleophile compound is 4-methylimidazole.

20- The process of any of claims 1 to 19, wherein the copper based catalyst is copper metal (0) or an organic or inorganic compound of copper (I) or copper (II).

21. The process according to claim 20, wherein the copper based catalyst is cuprous bromide, cupric bromide, cuprous iodide cuprous chloride, cupric chloride, basic copper (II) carbonate, cuprous nitrate, cupric nitrate, cuprous sulphate, cupric sulphate, cuprous sulfite, cuprous oxide, cupric oxide, cuprous acetate, cupric acetate, cupric trifluoromethylsulfonate, cupric hydroxide, copper (I) methylate, copper (II) methylate, cupric chloro methylate with the formula ClCuOCH₃.

22- The process of claim 20, wherein the copper based catalyst is a catalyst of a copper (I) basis, preferably, cuprous iodide, bromide or chloride and even better, cuprous iodide.

23- The process of any of claims 1 to 22, wherein the base has a pka that is at least more than or equal to 4, preferably between 4 and 14, and even better, between 4 and 10.

24- The process of claim 23, wherein the base is a mineral base, preferably alkaline metal carbonate, hydrogenocarbonate, phosphate, polyphosphate, borate or silicate, preferably, sodium, potassium; or a metal belonging to alkaline earths, preferably calcium, barium or magnesium, or a transition metal, preferably, zinc and copper.

25- The process of claim 23, wherein the base is an organic base, preferably triethylamine, tri-n-propylamine, tri-n-butylamine, methyldibutylamine, methyldicyclohexylamine, ethyldiisopropylamine, N,N-diethylcyclohexylamine, pyridine, dimethylamino-4 pyridine, N-methylpiperidine, N-ethylpiperidine, N-n-butylpiperidine, 1,2-dimethylpiperidine.

26- The process of claim 23, wherein the base is potassium carbonate.

27- The process of any of claims 1 to 26, wherein the organic solvent has a dielectric constant comprised between 2 and 15.

28- The process of any of claims 1 to 27, wherein that the organic solvent has a donor number that is between 4 and 25, preferably between 7 and 20.

29- The process of any of claims 1 to 28, wherein the organic solvent is an aliphatic, cycloaliphatic or aromatic ether-oxide.

30- The process of claim 29, wherein the organic solvent is diethyl oxide, dipropyl oxide, diisopropyl oxide, dibutyl oxide, methyl and tertiobutyl oxide, dipentyl oxide, diisopentyl oxide, ethyleneglycol dimethylether (or 1,2-dimethoxyethane), diethyleneglycol dimethylether (or 1,5-dimethoxy-3-oxapentane), anisole, 1,2-dimethoxybenzene, 1,3-dimethoxybenzene, 1,4-dimethoxybenzene, dioxane, tetrahydrofurane.

31- The process of claim 30 wherein the organic solvent is anisole.

32- The process of any of claims 1 to 31 wherein the extracting agent has the following formula: 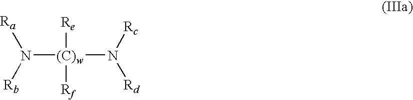
in the said formula:
Rₐ, R_{b}, R_{c}, R_{d}, identical or different, represent a hydrogen atom or an alkyl group,
Rₑ, R_{f}, identical or different, represent a hydrogen atom, an alkyl group or a phenyl group,
w lies between 0 and 6.

33- The process of claim 32, wherein the extracting agent satisfies formula (IIIa) in which Rₐ, R_{b}, R_{c}, R_{d}, identical or different represent a hydrogen atom or an alkyl group having from 1 to 4 hydrogen atoms and much preferably, Rₐ, R_{b}, R_{c}, R_{d}, represent a hydrogen atom.

34- The process of claim 32, wherein the extracting agent satisfies formula (IIIa) in which Rₑ and R_{f}, identical or different, represent a hydrogen atom or an alkyl group having from 1 to 4 hydrogen atoms, and more preferably, Rₑ and R_{f}, represent one hydrogen atom.

35- The process of claim 32, wherein the extracting agent satisfies formula (IIIa) wherein w lies between 2 and 4 and is preferably equal to 2.

36- The process of claim 32, wherein the extracting agent is 1,2-diaminoethane, 1,3-diaminopropane, 2,3-diaminobutane, N,N,N′,N′-tetramethyl-1,2-diaminoethane, N,N′-diisopropyl-1,2-diaminoethane, N,N′-dimethyl-1,2-diaminoethane, 2,3-diamino-2,3-dimethylbutane, 2,3-diamino-2,3-diphenylbutane.

37- The process of claim 36, wherein the extracting agent is 1,2-diaminoethane.

38- The process of any of claims 1 to 31, wherein the extracting agent satisfies the following formula:
HO-Ψ-G (IIIb)
in the said formula:
Ψ represents a linear or branched aliphatic chain having 2 to 20 carbon atoms,
G represents one of the following functional groups:
OH
NH₂
NHRₐ
NRₐR_{b}
in the said groups Rₐ, R_{b}, identical or different, represent an alkyl group.

39- The process of claim 38, wherein the agent satisfies formula (IIIb) wherein represents a linear or branched, divalent aliphatic group, preferably having 2 to 20 carbon atoms.

40- The process of claim 38, wherein the agent satisfies formula (IIIb) wherein represents a hydro carbonated chain that may optionally be interrupted by a heteroatom or functional group, preferably, oxygen or NH or NHRₐ (with Rₐ as defined earlier).

41- The process of claim 38 or 39, wherein the hydro carbonated chain carries one or several substituents, preferably a further OH group.

42- The process of claim 38, wherein that the agent satisfies formula (IIIb) wherein Ψ represents ethylenepropylene, isopropylene, butylene, isobutylene, pentylene, propanetri-1,2,3-yle, 3-oxypentylene, 3-iminopentylene group.

43- The process of any of claims 38 to 42, wherein the agent satisfies formula (IIIb) wherein functional group G is a NHRₐ or NRₐR_{b} group wherein the groups Rₐ, R_{b}, identical or different, represent an alkyl group having 1 to 4 carbon atoms and preferably, Rₐ, R_{b}, represent a methyl or ethyl group.

44- The process of any of claims 38 to 43, wherein the agent satisfies formula (IIIb) wherein functional group G is a hydroxyl group or an amino group.

45- The process of claim 38, wherein the agent is chosen from:
diol type agents such as:
ethyleneglycol,
1,3-propanediol,
1,2-propanediol,
1,2-butanediol,
1,4-butanediol,
1,3 butanediol,
diethyleneglycol,
glycerol,
aminoalcohol type agents such as:
ethanolamine,
N,N-dimethylethanolamine,
1-amino-2-propanol,
3-amino-1-propanol,
2-amino-1-propanol,
2-amino-1-butanol,
4-amino-1-butanol,
4-amino-2-butanol,
2-(ethylamino)ethanol,
2-amino-1,3-propanediol,
3-amino-1,2-propanediol,
2-amino-2-methyl-1,3-propanediol,
diethanolamine,
N-methyldiethanolamine,
3-methylamino-1,2-propanediol,
2-amino-1,3-butanediol.

46- The process of claim 45, wherein the extracting agent is ethyleneglycol or ethanolamine.

47- The process of any of claims 1 to 46, wherein the bonding reaction takes place at a temperature that is situated between 80° C. and 170° C., preferably, between 120° C. and 160° C., and even better, at about 140° C.

48- The process of any of claims 1 to 47, wherein the bonding reaction takes place under atmospheric pressure but preferably, under controlled atmosphere of inert gases, preferably, nitrogen.
